(19) United States
(12) Patent Application Publication
(10) Pub. No.: US 2009/0171229 A1
(43) Pub. Date: Jul. 2, 2009

(54) Swimmer's Starter Platform with Heart Rate Monitor

(76) Inventors: Thomas A. Saldarelli, Yorktown Heights, NY (US); Anita Sayed, Loveland, CO (US)

Correspondence Address: GREENBERG TRAURIG (PHX), INTELLECTUAL PROPERTY DEPARTMENT, 2450 COLORADO AVENUE , SUITE 400E, SANTA MONICA, CA, 90404, US

(21) Appl. No.: 12/202,047

(22) Filed: Aug. 29, 2008

Related U.S. Application Data
(60) Provisional application No. 60/966,588, filed on Aug. 29, 2007.

(51) Int. Cl. A61B 5/044; E04H 4/14

(52) U.S. Cl. 600/523; 4/496

(57) ABSTRACT

Some embodiments of the invention provide a swimmer's starter platform including a heart rate monitor. The heart rate monitor can include electrodes to acquire heart rate data and a transmitter. The starter platform can include a detachable display including a receiver. The detachable display can wirelessly receive the heart rate data and display the heart rate data whether coupled or detached from the starter platform. The starter platform can include a top platform, backstroke handlebars, and electrode handlebars. The electrode handlebars can include electrodes to acquire heart rate data. The electrode handlebars can be positioned with respect to the top platform and the backstroke handlebars to acquire the heart rate data while the swimmer is in the pool.

### RELATED APPLICATIONS

This application claims priority to U.S. Provisional Application No. 60/966,588, filed Aug. 29, 2008, the entire contents of which are incorporated herein by reference.

### BACKGROUND

Heart rate is a common physiological measurement that athletes use to track their physical fitness and capabilities. Monitoring measurements for resting heart rate, target heart rate, and maximum heart rate can allow an athlete to receive the most beneficial workout for their own health. Specifically, training in an optimal cardiac state can offer quicker improvements seen in physical fitness in comparison to workouts performed while outside the target heart rate.

Monitors on common exercise apparatuses like treadmills, stationary bikes, elliptical machines, and even jump ropes can accurately measure heart rates to allow runners, joggers, and bikers to track their physiological condition during workouts. These apparatuses contain electrically conductive surfaces, usually on handlebars, to retrieve heart rate signals from the body and display the information to the athlete.

Unfortunately, an accurate and convenient method for measuring heart rate for swimmers has not yet been attainable. The constant motion as well as total immersion in water greatly inhibits conventional heart rate measurement. “Waterproof” electrode assemblies have been created for this purpose. However, the relay of information becomes an issue when exercise includes laps across a pool. The use of connective cables is inhibiting to a swimmer immersed in the water as well as unpractical as data loss due to leakage can occur across long cables. Further, wireless assemblies have difficulty differentiating between actual signal and noise when attempting data transfer across such a long range. In addition, all of these inventions have required additional apparatuses outside the pool for data processing and display. Despite these inventions, what is still needed is an accurate method of measurement and a convenient apparatus for processing and displaying information.

Swimmer's starter platforms in recent years have become more than just blocks for take off. Inventors have realized the convenience of the platform within a pool area and thus starter platforms now can contain timing devices, lap counters, pressure sensors for accurate take-off, and even light-based starting systems. While these systems can help track overall physical fitness, like speed and reaction time, none have the capacity to track cardiovascular endurance.

### SUMMARY

Some embodiments of the invention provide a swimmer's starter platform including a heart rate monitor. The heart rate monitor can include electrodes to acquire heart rate data and a transmitter. The starter platform can include a detachable display including a receiver. The detachable display can wirelessly receive the heart rate data and display the heart rate data whether coupled to the starter platform or detached from the starter platform.

Some embodiments of the invention provide a starter platform including a top platform, backstroke handlebars, and electrode handlebars. The electrode handlebars can include electrodes to acquire heart rate data. The electrode handlebars can be positioned with respect to the top platform and the backstroke handlebars to acquire the heart rate data while the swimmer is in the pool. The starter platform can display the heart rate data.

Other aspects of the invention will become apparent by consideration of attached the description and accompanying drawings.

### BRIEF DESCRIPTION OF THE DRAWINGS

FIG. 1 is a front view of a swimmers' starter platform with a heart rate monitor according to one embodiment of the invention.

FIG. 2 is a side view of the swimmers' starter platform with a heart rate monitor of FIG. 1.

FIG. 3 is a front view of the heart rate monitor of the swimmers' starter platform of FIGS. 1 and 2.

FIG. 4 is a side view of the heart rate monitor of FIG. 3.

FIG. 5 is a schematic diagram of the heart rate monitor of FIGS. 3 and 4.

FIG. 6 is a front view of a display for use with the heart rate monitor of FIGS. 1-5.

FIG. 7 is a side view of the display of FIG. 6.

FIG. 8 is a front view of a display including controls for use with the heart rate monitor of FIGS. 1-5.

FIG. 9 is a detailed front view of the display of FIG. 8.

FIG. 10 is a detailed side view of the display of FIG. 8.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

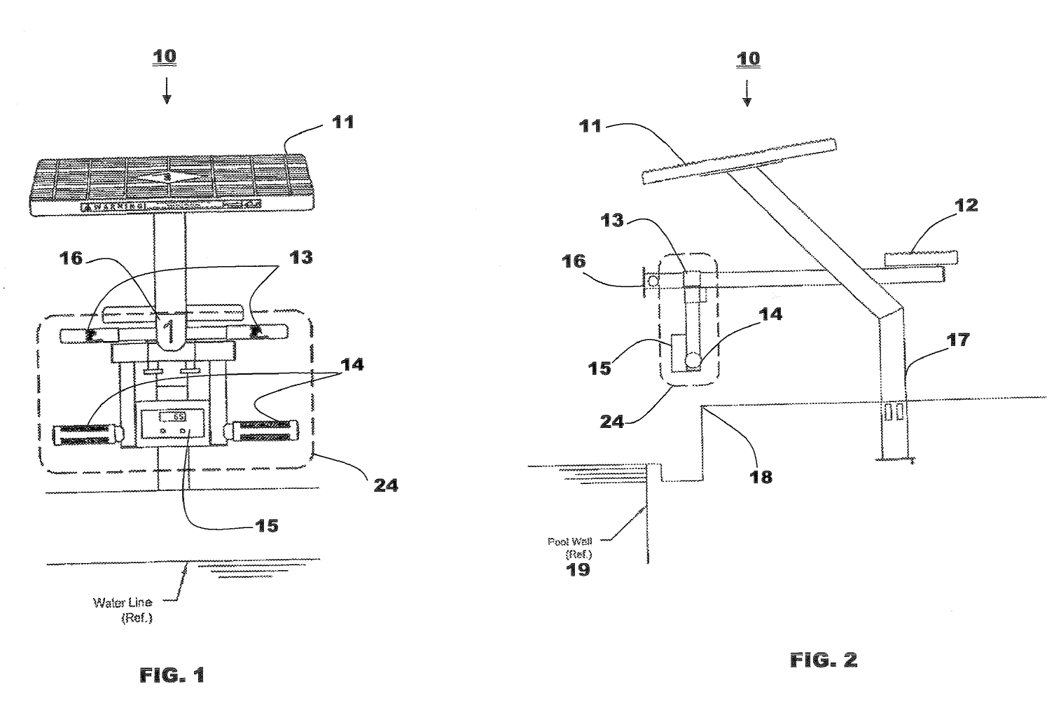

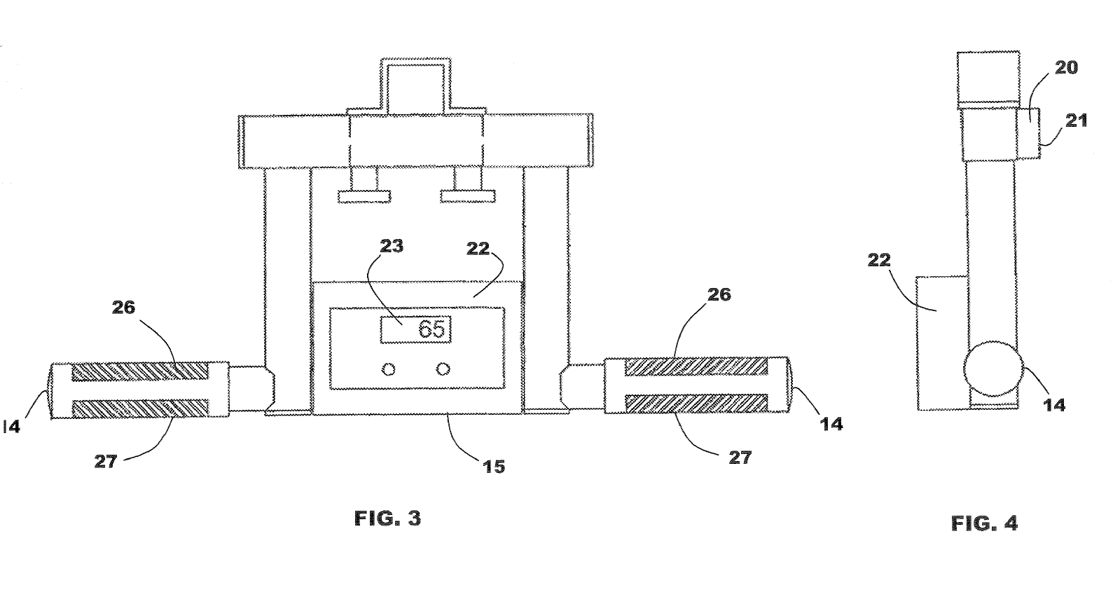

FIGS. 1 and 2 illustrate a front and side view of a swimmer's starter platform 10 (also known as a starting block) according to one embodiment of the invention. The swimmer's starter platform 10 can include a top platform 11, at least one step 12, backstroke handlebars 13, a heart rate (pulse) monitor 24 including electrode handlebars 14 and an LCD display and controls device 15, a lane number 16, and a mounting structure 17. The basic structure of all elements on the starter platform 10 can be in accordance with respective swimming associations rules and regulations (e.g., Federation Internationale de Natation). The mounting structure 17 can be made of strong material to mount the platform 10 adjacent to the pool. The top platform 11 can be made of a firm material that does not allow for any spring effect from forces such as swimmers diving off and can be covered with a non-slip material. The step 12 can be included directly behind or beside the top platform 11. The backstroke handlebars 13 can be coupled to the mounting structure 17 in line with the pool edge 18 or pool wall 19. The backstroke handlebars 13 can either be oriented horizontally (parallel to the water line, as shown in FIG. 1) or vertically (perpendicular to the water line). The electrode handlebars 14 can also be coupled to the mounting structure 17 similar to the backstroke handlebars 13. The electrode handlebars 14 can be substantially lower than the backstroke handlebars 13 so that a swimmer in the water can grip the electrode handlebars 14 without strained effort to hold himself out of the water. In addition, the electrode handlebars 14 can sit slightly further back from the backstroke handlebars 13 with respect to the pool wall 19. While the electrode handlebars 14 can support a swimmer's weight, they are not intended, in this embodiment, to replace the backstroke handlebars 13. An LCD display and controls device 15 can be coupled to the mounting structure 17 facing the pool so that a swimmer can view the display or it can have the ability to rotate 90 degrees left or right so someone poolside can also view the display.

The electrode handlebars 14 can be positioned as shown in FIG. 1 for a swimmer to grasp/grip the electrode handlebars 14 while in the water. The electrode handlebars 14 can be positioned parallel to the backstroke handlebars 13, as shown, or perpendicular to the backstroke handlebars 13. With this configuration, heart rates can be measured at different periods of activity, including resting, active, and recovery: resting heart rates can be measured prior to starting a workout, periodic active heart rates can be measured at time intervals during a workout to monitor the swimmer's heart rate range during exercise, and recovery heart rates can be measured immediately after a workout to monitor recovery time. The heart rate data from these measurements can be obtained quicker as the swimmer does not need to leave the pool. As a result, the heart rate data can be more accurate relative to the desired activity period.

In some embodiments, the electrode handlebars 14 can be located near the top platform 11, so a swimmer can grasp the electrode handlebars 14 prior to diving into the pool. Therefore, a resting heart rate can be measured prior to a race while the swimmer is on the starting platform 10 or active heart rates can be measured during training that requires the swimmer to continuously dive off the starting platform 10 rather than start from inside the pool.

FIGS. 3 and 4 illustrate the heart rate monitor 24 that is coupled to the starter platform 10 according to one embodiment of the invention. A swimmer's heart rate can be identified by signals detected from the electrode handlebars 14. Accordingly, each of the electrode handlebars 14 can contain at least two electrodes (top electrode 26 and bottom electrode 27) made of stainless steel, conductive rubber, or similar material that provides high conductivity and resistance to oxidation. The heart rate can be detected when the swimmer's palm of each hand is in contact with each top electrode 26 and the fingertips of each hand are in contact with each bottom electrode 27. Information from the electrodes can be transferred to a wireless transmitter 20 within a transmitter enclosure box 21 by a direct connection (cables, wires, etc. within the handlebars can be routed directly to the transmitter), in some embodiments. The wireless transmitter 20 can send a signal to a wireless receiver 22 coupled to the LCD display and controls device 15. The signal can then be processed and the heart rate can be displayed on the LCD display 23.

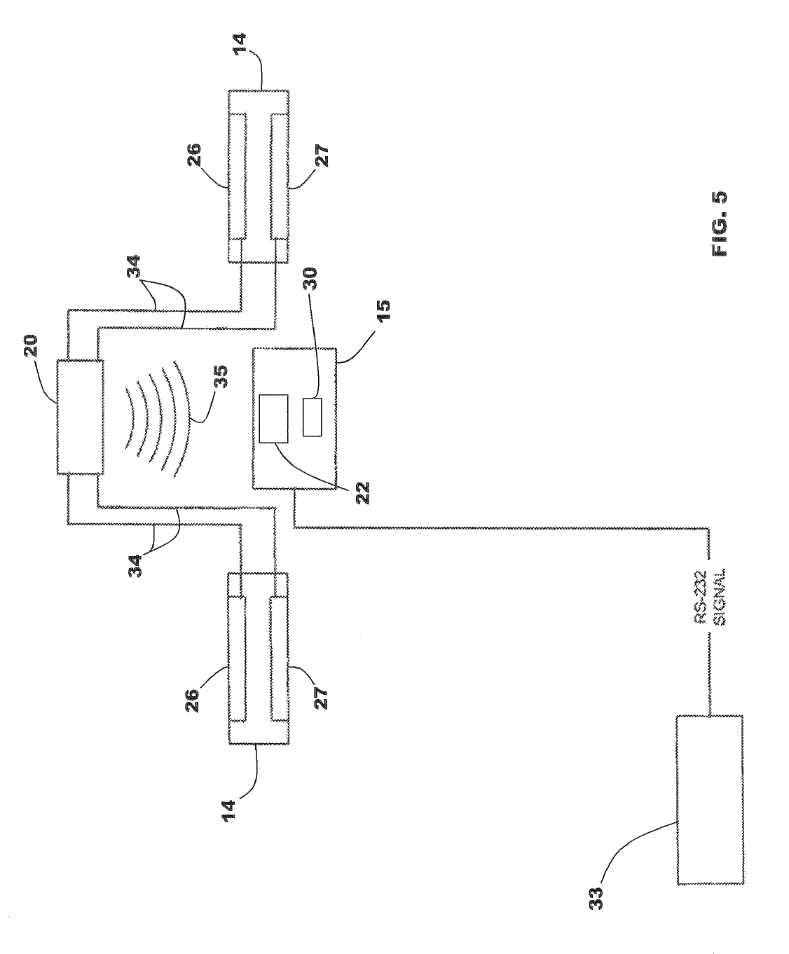

FIG. 5 is a schematic diagram of the heart rate monitor 24 shown and described in FIGS. 3 and 4. When the swimmer's hands are in contact with the electrode handlebars 14 (i.e., the swimmer's palm is in contact with top electrode 26 to obtain the heart rate signal, and the swimmer's fingertips are in contact with bottom electrode 27 as a reference), the heart rate is detected. The heart rate signal is routed to the wireless transmitter 20 via wires 34. The transmitter 20 sends a signal at some frequency (e.g., 5 kHz) that can be received by the wireless receiver 22. The locations of the transmitter 20 and the receiver 22 relative to each other can be within an optimal range for wireless transmission. The display and controls device 15 can include a microprocessor 30. The microprocessor 30 can store algorithms to process an average of the swimmer's heart rate over a period of time (e.g. 10 seconds) and the average can be displayed on the LCD display 23, in some embodiments. In addition, some embodiments include a microprocessor within the transmitter enclosure box 21, as shown in FIG. 4, to process the information prior to transmission.

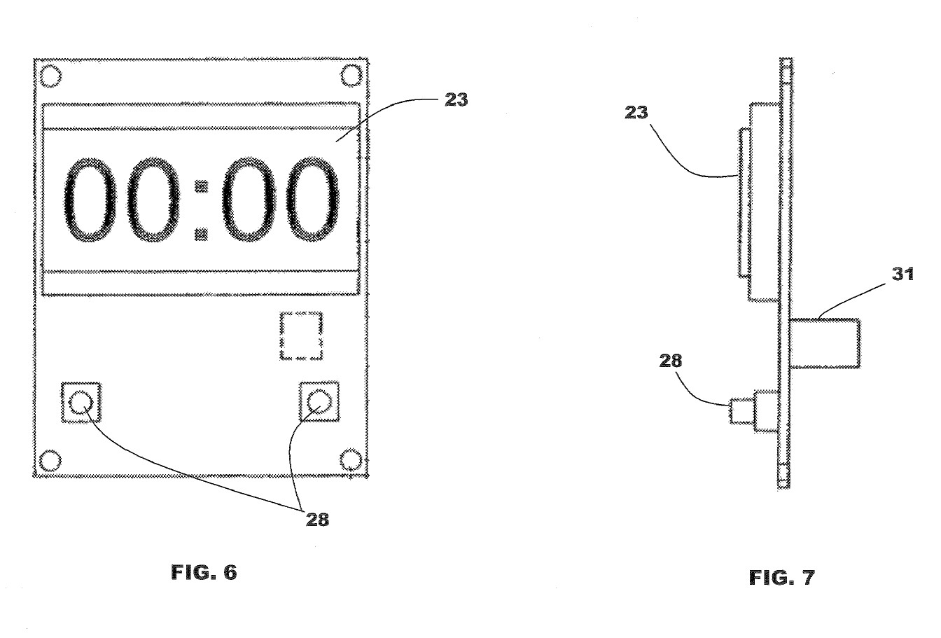

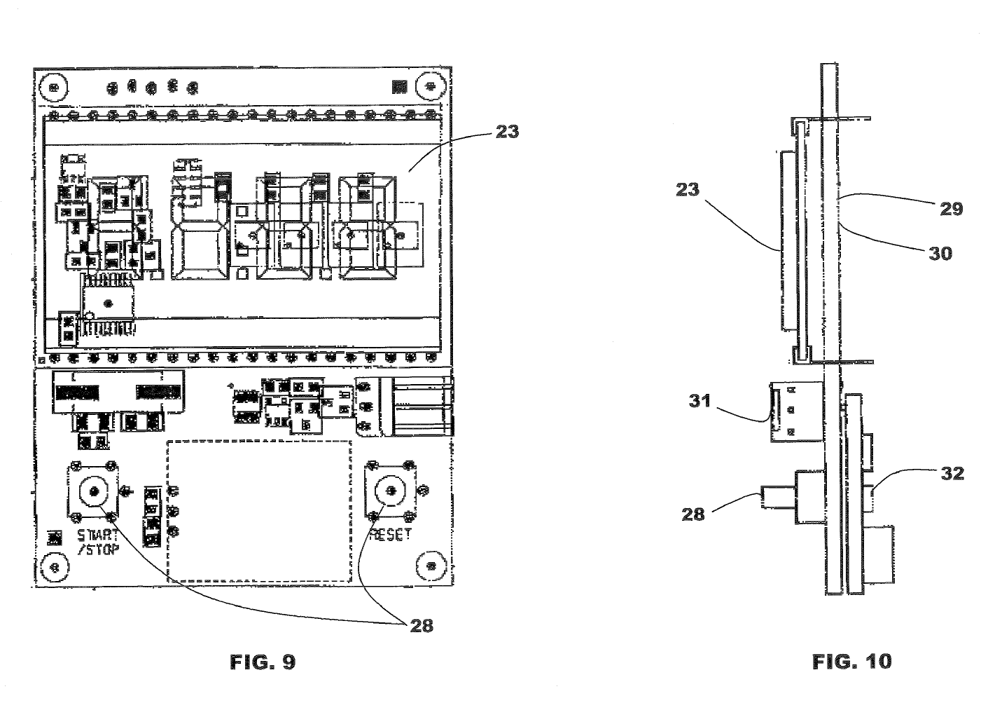

In some embodiments of the invention, the electrical and mechanical specifications of the LCD display and controls device 15 can include the details illustrated in FIGS. 6-10. In one embodiment, the user interface can include a 4-digit, 7-segment LCD display 23. In addition, mechanical switches 28 can accompany the display 23 for user control. As shown in FIG. 10, a printed circuit board 29 can include circuitry, such as a microprocessor 30. In one embodiment, the microprocessor 30 can be a 400 kHz, 8 bit processor with on-chip RAM and can include input/output capabilities, timers, RS-232 compatibility, and 8 KB flash program storage. Power requirements of the microprocessor 30 and the LCD display and controls device 15 can be supported by a battery and/or the power connector 31. Additionally, to manage noise within the system, a filter can also be installed within the LCD display and controls device 15 to allow only one pulse per a designated time interval (e.g., 250 ms), allowing a more accurate representation of heart rate.

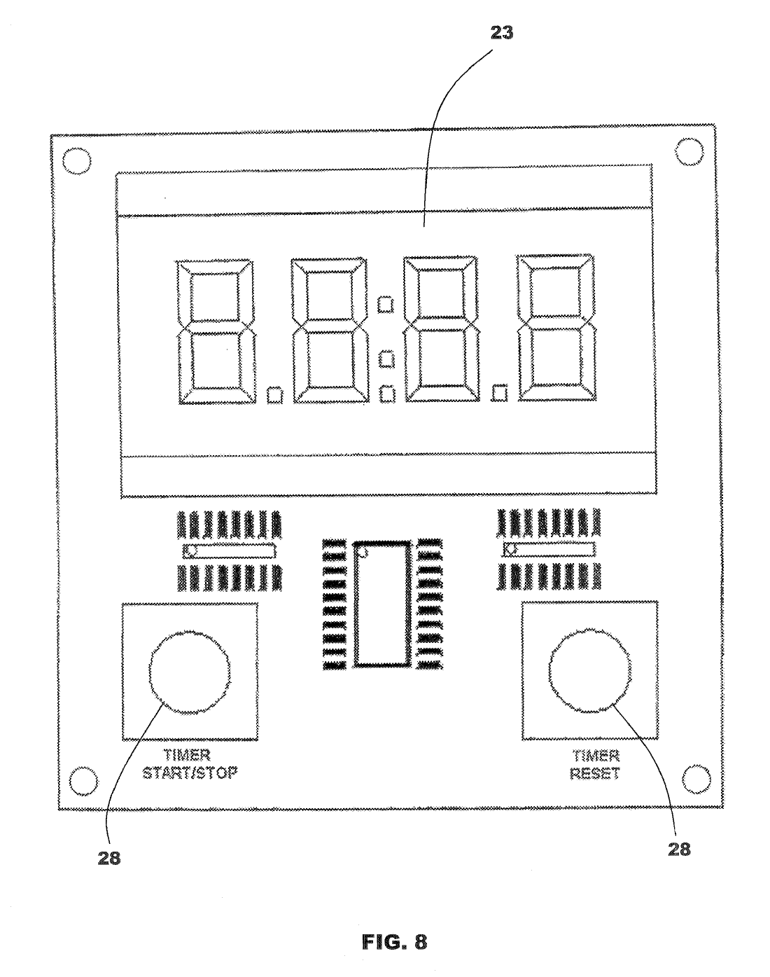

The LCD display 23 can be used for heart rate output from the microprocessor 30 or for a simple timer in some embodiments of the invention. Time can be displayed in the format 00:00 as shown in FIGS. 6 and 8. Therefore, time can count up to 99:59 (99 minutes and 59 seconds). The LCD display and controls device 15 can have an automatic power-down feature to conserve energy if accidentally left on. If heart rate is being detected, the display 23 can toggle between time and heart rate at set time intervals (e.g., every 10 seconds). Heart rate can be displayed in the format 000 as beats per minute. In addition, when heart rate is detected, a decimal point on the display 23 can flash each time a pulse is received.

As shown in FIG. 8, mechanical switches 28 can be used for a “start/stop” option and a “reset” option in the form of pushbuttons, in some embodiments of the invention. The “start/stop” switch can be used to turn on the device, initiate the accumulation of time, pause the time count, and resume the time count. If the timer has been paused and heart rate is being monitored, the display 23 can continue to toggle between showing accumulated time and heart rate. The “reset” switch can be used while the LCD display and controls device 15 is on to set the accumulated time to 00:00 on the display 23. Whether the display 23 is showing time or heart rate, once the “reset” switch is depressed, the display 23 can automatically show 00:00. In addition, pressing and holding the “reset” switch for some time interval (e.g., 3 seconds) can turn off power to the display 23.

The physical connection made between the LCD display and controls device 15 and the mounting structure 17 can allow for up to a 180-degree rotation of the device to allow coaches or officials poolside to view the display 23 or access the mechanical switches 28. In some embodiments, the LCD display and controls device 15 can be removable from the mounting structure 17 and can use wireless technology to communicate with the wireless transmitter 20. A removable/detachable LCD display and controls device 15 can allow persons to view the display 23 or access the mechanical switches 28 while it is not physically connected to the starter platform 10, but is located within range of the wireless transmitter. This detachable feature can be of use for a coach or official monitoring heart rate during a race or training session.

In addition to the LCD display 23, some embodiments of the invention can include a RS232 serial port to output the heart rate information data to other devices. For example, FIG. 5 illustrates the RS232 signal used to output the heart rate data to a scoreboard display 33. However, this operation is not limited to a scoreboard display; any device with an RS232 serial port can receive the heart rate data from the starter platform 10. While an RS232 port is suitable, alternative embodiments can use other forms of serial communication.

FIG. 10 also illustrates a receiver board 32, which can be included in some embodiments of the invention. The receiver board 32 can be used in conjunction with a chest belt to transmit heart rate information while the swimmer is not in contact with the starter platform 10 via wireless signal transmission This can be incorporated into the platform 10 to allow active heart rate as well as resting heart rate measurement capabilities within the same system.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A swimmer's starter platform comprising:
a heart rate monitor including at least two electrodes to acquire heart rate data and a transmitter; and
a detachable display coupled to the starter platform, the detachable display including a receiver,
the detachable display wirelessly receiving the heart rate data and displaying the heart rate data,
the detachable display displaying heart rate data while coupled to the starter platform,
the detachable display displaying heart rate data while detached from the starter platform.

2. The swimmer's starter platform of claim 1, wherein the detachable display, while coupled to the starter platform, is rotatable.

3. The swimmer's starter platform of claim 1, wherein the detachable display displays heart rate data in the form of a digital numeric display corresponding to measured beats per minute

4. The swimmer's starter platform of claim 1, wherein the detachable display displays heart rate data in the form of a point flashing with respect to at least one beat measured.

5. The swimmer's starter platform of claim 1, wherein the detachable display displays elapsed time.

6. The swimmer's starter platform of claim 1, wherein the detachable display includes a serial port to transmit heart rate data.

7. A starter platform for use by a swimmer, the starter platform comprising:
a top platform;
at least two backstroke handlebars;
at least two electrode handlebars including at least two electrodes to acquire heart rate data, the at least two electrode handlebars positioned with respect to the top platform and the backstroke handlebars to acquire the heart rate data while the swimmer is in the pool; and
a display coupled to the starter platform displaying the heart rate data.

8. The starter platform of claim 7, wherein the at least two electrode handlebars are positioned below the at least two backstroke handlebars to acquire heart rate data from the swimmer while the swimmer is in the pool during at least one of a rest period and a recovery period.

9. The starter platform of claim 7, wherein the at least two electrode handlebars are positioned above the at least two backstroke handlebars and below the top platform to acquire heart rate data from the swimmer while the swimmer in on the top platform.

10. The starter platform of claim 7, wherein the at least two electrode handlebars are positioned parallel to the at least two backstroke handlebars.

11. The starter platform of claim 7, wherein the at least two electrode handlebars are positioned perpendicular to the at least two electrode handlebars.

12. The starter platform of claim 7, wherein the heart rate data is transmitted to and displayed on a scoreboard.

13. A method of acquiring heart rate data of a swimmer at a pool, the method comprising:
providing a starter platform including at least two electrode handlebars, a transmitter, and a detachable display, the detachable display including a receiver and a controls device;
acquiring heart rate data from the swimmer while the swimmer grips the at least two electrode handlebars;
transmitting the heart rate data from the at least two electrode handlebars to the transmitter;
transmitting the heart rate data from the transmitter to the receiver of the detachable display,
processing the heart rate data with the controls device of the detachable display; and
displaying the heart rate data on the detachable display.

14. The method of claim 13, wherein the transmitting of heart rate data from the electrode handlebars to the transmitter is performed via physical wire connections.

15. The method of claim 13, wherein the transmitting of heart rate data from the transmitter to the receiver is performed via a wireless connection.

16. The method of claim 13, wherein the acquiring of heart rate data from the swimmer while the swimmer grips the at least two electrode handlebars occurs while the swimmer is in the pool.

17. The method of claim 13, wherein the acquiring of heart rate data from the swimmer while the swimmer grips the at least two electrode handlebars occurs while the swimmer on top of the starter platform.

18. The method of claim 13, wherein the displaying of the heart rate data on the detachable display occurs while the detachable display is coupled to the starter platform.

19. The method of claim 13, wherein the displaying of the heart rate data on the detachable display occurs while the detachable display is detached from the starter platform.

20. The method of claim 13, and further comprising displaying the heart rate data on a scoreboard.
